(19) United States
(12) Patent Application Publication
(10) Pub. No.: US 2022/0249648 A1
(43) Pub. Date: Aug. 11, 2022

(54) RECOMBINANT BACULOVIRUS DISPLAYING AFRICAN SWINE FEVER VIRUS PROTEINS, AND AN IMMUNOLOGICAL COMPOSITION COMPRISING THE SAME

(71) Applicants: Ming-Che Shih, Culver City, CA (US); Academia Sinica, Taipei (TW)

(72) Inventors: Yu-Chan Chao, Taipei (TW); Wei-Ting Hsu, Taipei (TW)

(21) Appl. No.: 17/611,925

(22) PCT Filed: Jul. 31, 2020
(86) PCT No.: PCT/US2020/044411

§ 371 Date: Nov. 17, 2021

Related U.S. Application Data
(60) Provisional application No. 62/881,988, filed on Aug. 2, 2019.

(51) Int. Cl. A61K 39/12; A61P 31/12; A61P 37/04; A61K 47/69; G01N 33/569

(52) U.S. Cl. CPC A61K 39/12; A61P 31/12; A61K 2039/5256; A61K 47/6901; G01N 33/56983; A61P 37/04

(57) ABSTRACT

Provided are a vector, a recombinant virus, and a method of using and making thereof. Also provided are immunological compositions containing the recombinant African swine fever virus (ASFV) for inducing an immunological response in a host animal to which the immunological composition is administered. Further provided is a kit and a method of detecting the presence of ASFV immunogens in a sample from an animal.

### BACKGROUND

### 1. Technical Field

The disclosure relates to vectors and viruses, and to methods of making and using the same. The disclosure further relates to recombinant vectors that express gene products of interest and the recombinant viruses obtained therefrom, and to the cells or insects infected, transformed or transfected with such vectors and viruses. Moreover, the disclosure is also directed to such vectors and viruses that induce an immune response directed to or against African swine fever virus (ASFV) and such compositions that are immunological and immunogenic, or vaccine compositions that confer protective immunity against infection by ASFV. The disclosure yet further relates to the uses of and methods for making and using such vectors and compositions, as well as the products therefrom, such as methods and kits for detecting ASFV.

### 2. Description of Related Art

Swine provides an important source of high-quality proteins and contributes an important share in the animal husbandry and economy. However, the swine industry has been under threats with the epidemic of several infectious diseases. Amongst these diseases, African swine fever (ASF) is currently causing greatest concern. This is particularly true since its introduction to and dramatic spreading in 2018 in China, a major consumer of pork where half of the world's swine population is raised. African swine fever virus (ASFV) causes rapid death of almost all infected pigs and wild boar. The lack of a vaccine hinders control, which is further complicated by the presence of infected wild suids in some regions. As ASF is a notifiable disease to the World Organization for Animal Health (OIE), introduction to a new country or region results in imposition of trade restrictions and therefore may cause serious economic losses. Attempts to control the disease require international cooperation, and there is a huge unmet need in the development of vaccines and other control strategies.

The African swine fever virus (ASFV) is an enveloped virus belonging to the genus Asfivirus of Asfarviridae family. The genome consists of a linear dsDNA molecule of 170 to 190 kb with terminal inverted repetitions. The viral genome encodes for more than 50 structural proteins and several non-structural proteins. Many viral proteins have been expressed and tested for the protection of the pigs against the infection of ASFV. However, these prior developed vaccines either failed or only partially protected the pigs. It has been shown that in comparison to subunit vaccines, the live attenuated viruses are shown to be the most effective. However, these vaccines result in chronic ASFV infection, and demonstrated side-effects including pneumonia, abortion, locomotor disturbances, necrotic foci, and even death. Therefore, an effective and safe vaccine is urgently needed.

### SUMMARY

In one aspect, this disclosure provides compositions and methods for treatment and prophylaxis of infection with ASFV.

In another aspect, the present disclosure relates to an antigenic, immunological, immunogenic, or vaccine composition or a therapeutic composition for inducing an antigenic, immunogenic or immunological response in a host animal inoculated with the composition. The composition comprising a recombinant virus, such as a baculovirus, and relates to displaying ASFV proteins either on the envelope of the baculovirus or fused with the baculovirus capsid protein. Displaying ASFV proteins or protein subunits on the baculovirus surface retain their natural conformations and enhance the immunogenicity of the displayed ASFV proteins or protein subunits.

In still another aspect, the baculovirus as the carrier in the present disclosure to carry and display the ASFV protein or protein subunit also serve as an adjuvant itself to boost the effect of the displayed proteins or protein subunits. Furthermore, displaying by baculovirus reduces the efforts for purification of the protein subunits, since the baculovirus buds out of the infected cells, and the vaccine antigens can be collected from the medium without extensive efforts.

The present disclosure therefore discloses a recombinant baculovirus having at least one of the ASFV proteins P72, P49, PE120R, P54, P30 and CD2v, or a combination thereof.

In one embodiment, the recombinant baculovirus comprises an ASFV protein that is fused with a baculovirus protein or a fragment thereof. In one embodiment, the ASFV protein is displayed on the surface of the recombinant baculovirus, where the surface is a baculoviral capsid or envelope. In another embodiment, the ASFV protein fused with a baculovirus protein or a fragment thereof is displayed on the surface in its natural conformation. In another embodiment, at least one of the ASFV proteins P72, P49 and PE120R is displayed on the surface of the baculoviral capsid. In yet another embodiment, at least one of the ASFV proteins P54, P30 and CD2v is displayed on the surface of the baculoviral envelope. In one embodiment, the baculovirus protein or a fragment thereof fused with the ASFV protein is a capsid protein or an envelope protein. In another embodiment, the baculovirus protein or a fragment thereof fused with the ASFV protein is VP39, Fusion (F) protein or GP64.

In one embodiment, the recombinant baculovirus comprises at least one of the ASFV proteins P72, P49 and PE120R displayed on the baculoviral capsid and at least one of the ASFV proteins P54, P30 and CD2v displayed on the baculoviral envelope simultaneously.

In another embodiment, the recombinant baculovirus expresses an adjuvant protein. In yet another embodiment, the adjuvant protein is granulocyte-macrophage colony-stimulating factor (GMCSF), chemokine C-C motif ligand 25 (CCL25) or chemokine C-C motif ligand 29 (CCL29).

The present disclosure also relates to a cloning vector that produces the recombinant baculovirus.

Another aspect of the present disclosure relates to an immunological composition comprising the recombinant baculovirus as mentioned above, and further relates to a method for inducing an immunological response against ASFV in a host capable of producing an immunological response against ASFV comprising administering to the host the immunological composition.

In yet another aspect, the present disclosure relates to a cell infected with the recombinant baculovirus as mentioned above. In one embodiment, the cell displays the ASFV protein on the cell surface. In another aspect, the present disclosure relates to a method for detecting ASFV in a host comprising detecting formation of a complex between the cell and a sample obtained from the host. The detection is performed by immunoassay, counter immuno-electrophoresis, radioimmunoassay, radioimmunoprecipitation assay, enzyme-linked immunosorbent assay (ELISA), dot blot assay, inhibition of competition assay or sandwich assay. The sample obtained from the host can be any biological substance that contains the immunogenic molecule, such as serum or antiserum. In one embodiment, the host is a porcine.

In another aspect, the present disclosure relates to a kit comprising one or more of the cells mentioned above and a reagent for immunological detection.

### BRIEF DESCRIPTION OF THE DRAWINGS

The present disclosure will become more readily appreciated by reference to the following descriptions in conjunction with the accompanying drawings below.

FIG. 1 shows the organization of the expression cassette and the construction maps of the plasmids, pTriEx-VP39-P72, pTriEx-P72-VP39, pTriEx-P72, pTriEx-P54, pTriEx-CD2v, pTriEx-P30, and pTriEx-GMCSF, for generation of membrane-anchored recombinant baculovirus, VP39-P72-Bac, P72-VP39-Bac, P72-Bac, P54-Bac, CD2v-Bac, P30-Bac and GMCSF-Bac, respectively. TM: baculovirus envelope glycoprotein 64 (GP64) transmembrane domain (TM); CTD: the GP64 cytoplasmic tail domain (CTD); SP: honeybee melittin signal peptide; 6H: 6×His-tag.

FIGS. 2A to 2G show the western blots of the ASFV VP39-P72 (FIG. 2A), P72-VP39 (FIG. 2B), P72 (FIG. 2C), P54 (FIG. 2D), CD2v (FIG. 2E), P30 (FIG. 2F), and GMCSF (FIG. 2G) proteins detected in the cell lysate of VP39-P72-Bac, P72-VP39-Bac, P72-Bac, P54-Bac, CD2v-Bac, P30-Bac and GMCSF-Bac infected Sf21 cells at 3 days post infection with a multiplicity of infection (MOI) of 5. N: negative control; 1 to 13: cell lysates obtained with different constructs; wt: cell lysate from Sf21 cell infected with wild-type AcMNPV virus.

FIGS. 3A to 3C show the cell-based ELISA for detection of ASFV antibodies and antisera. FIG. 3A shows the ELISA read with anti-His antibody; FIG. 3B shows the ELISA read with ASFV pig serum; and FIG. 3C shows the ELISA read with the control serum. From left to right: P72, VP39-P72, P72-VP39, P30, P54 and wt (wild-type).

### DETAILED DESCRIPTIONS

In one aspect, the present disclosure relates to a recombinant virus, such as a recombinant baculovirus, containing therein a nucleotide sequence from ASFV. According to the present disclosure, the recombinant baculovirus expresses gene products of the foreign ASFV genes. Specific sequences encoding the antigenic proteins of ASFV are inserted into the baculovirus vector, and the resulting recombinant baculovirus is used to infect an animal Expression products of ASFV genes in the cells or animals result in an immune response to ASFV in the animal Thus, the recombinant baculovirus of the present disclosure may be used in an immunological composition or vaccine to induce an immune response in a subject in need thereof.

The disclosure also encompasses vectors encoding and expressing equivalent nucleotide sequences, e.g., the sequences which change neither the functionality nor the immunogenicity of the gene considered or those of the polypeptides encoded by this gene. The sequences differing through the degeneracy of the code are included. In one embodiment, the sequences are codon optimized for insect cells.

The nucleotide sequences used in the examples are derived from public database. For example, P72 is obtained from GenBank Accession No. MH722357.1; P54 is obtained from GenBank Accession No. MH735140.1; CD2v is obtained from GenBank Accession No. MH735142.1; P30 is obtained from GenBank Accession No. MH735141.1; and GMCSF is obtained from GenBank Accession No. U67175.1. These sequences, or fragments thereof, or regions thereof encoding an antigen protein or epitope of interest can also be used in this disclosure.

The disclosure also encompasses the equivalent sequences to those used herein and in documents cited herein, e.g., sequences that are capable of hybridizing to the nucleotide sequence under high stringency conditions (see, e.g., Sambrook et al. (1989)). Among the equivalent sequences, there may also be mentioned the gene fragments conserving the immunogenicity of the complete sequence, e.g., an epitope of interest.

Before the embodiments of the present disclosure are described in further details, it shall be noted that as used herein and in the appended claims, the singular forms “a,” “an,” and “the” include plural referents, unless the context clearly dictates otherwise.

Unless defined otherwise, all technical and scientific terms used herein have the same meanings as commonly understood by one of ordinary skill in the art, to which this disclosure belongs. All given ranges and values may vary by 1% to 5%, unless indicated otherwise or known otherwise by a person skilled in the art. Therefore, the term “about” is usually omitted from the description and claims. It should be understood that any methods and materials similar or equivalent to those described herein can be used in the practice or testing of the present disclosure. All publications mentioned herein are incorporated herein by reference for the purpose of describing and disclosing the substances, excipients, carriers, and methodologies as reported in the publications which might be used in connection with this disclosure. Nothing herein is to be construed as an admission that this disclosure is not entitled to antedate such disclosure by virtue of prior disclosure.

The present disclosure will employ, unless otherwise indicated, conventional techniques of molecular biology, microbiology, recombinant DNA technology, protein chemistry and immunology, which are within the skill of the art. Such techniques are explained fully in the pertinent literature.

In the context of the present disclosure, the term “immune response” or “immunological response” means, but is not limited to, the development of a cellular and/or antibody-mediated immune response to the one or more ASFV as described and/or defined herein or the composition or immunogenic composition or vaccine as described and/or defined herein. Usually, an immune or immunological response includes, but is not limited to, one or more of the following effects: the production or activation of antibodies, B cells, helper T cells, suppressor T cells, and/or cytotoxic T cells, directed specifically to an antigen or antigens included in the one or more ASFV as described and/or defined herein or the composition or immunogenic composition or vaccine as described and/or defined herein. The host will display either a therapeutic or a protective immunological (memory) response such that resistance to new infection will be enhanced and/or the clinical severity of the disease will be reduced. Such protection will be demonstrated by either a reduction in number of symptoms, severity of symptoms, or the lack of one or more of the symptoms associated with the infection of the wild-type ASFV, a delay in the onset of viremia, reduced viral persistence, a reduction in the overall viral load and/or a reduction of viral excretion.

In the context of the present disclosure, the term “effective dose” means, but is not limited to, an amount of antigen that elicits, or is able to elicit, an immune response that yields a reduction of clinical symptoms in an animal, to which the antigen is administered.

In the context of the present disclosure, the term “effective amount” means, in the context of a composition, an amount of an immunogenic composition capable of inducing an immune response that reduces the incidence of, or lessens the severity of infection or incident of disease in an animal. For example, an effective amount refers to a plaque forming unit (pfu) per dose. Alternatively, in the context of a therapy, the term “effective amount” refers to the amount of a therapy which is sufficient to reduce or ameliorate the severity or duration of African swine fever, or one or more symptoms thereof, prevent the advancement of such disease, cause the regression of such disease, prevent the recurrence, development, onset, or progression of one or more symptoms associated with such disease, or enhance or improve the prophylaxis or treatment of another therapy or a therapeutic agent.

In some embodiments, the immunogenic composition of the present disclosure contains an adjuvant. “Adjuvants” as used herein can include any substance that enhances the immunological response in the host in addition to the antigen protein. In one embodiment, it includes displaying immunological factors and interleukins such as GMCSF (or GM-CSF, granulocyte-macrophage colony-stimulating factor), CCL25 (chemokine (C-C motif) ligand 25) or CCL29 (chemokine (C-C motif) ligand 29) on the recombinant baculovirus surface. In other embodiments, the adjuvant includes aluminum hydroxide, aluminum phosphate, and saponins, e.g., Quil A, QS-21 (Cambridge Biotech Inc., Cambridge Mass.), GPI-0100 (Galenica Pharmaceuticals, Inc., Birmingham, Ala.), water-in-oil emulsion, oil-in-water emulsion, and water-in-oil-in-water emulsion. The emulsion can be based on light liquid paraffin oil (European Pharmacopeia type); isoprenoid oil such as squalane or squalene; oil resulting from oligomerization of alkenes, e.g., isobutene or decene; esters of acids or of alcohols containing a linear alkyl group, e.g., plant oils, ethyl oleate, propylene glycol di-(caprylate/caprate), glyceryl tri-(caprylate/caprate) or propylene glycol dioleate; esters of branched fatty acids or alcohols, e.g., isostearic acid esters. The oil is used in combination with emulsifiers to form the emulsion. The emulsifiers may be nonionic surfactants, e.g., esters of sorbitan, of mannide (e.g., anhydromannitol oleate), of glycol, of polyglycerol, of propylene glycol and of oleic, isostearic, ricinoleic or hydroxystearic acid, which are optionally ethoxylated, and polyoxypropylene-polyoxyethylene copolymer blocks, e.g., the Pluronic products, such as L121.

The immunogenic compositions and/or vaccines as described and/or defined herein may be formulated using techniques similar to those used for other pharmaceutical compositions. Thus, the adjuvant and the one or more ASFV as described and/or defined herein may be stored in lyophilized form and reconstituted in a physiologically acceptable vehicle to form a suspension prior to administration. Alternatively, the adjuvant and the one or more ASFV as described and/or defined herein may be stored in the vehicle. In some embodiments, vehicles are sterile solutions, e.g., sterile buffer solutions, such as phosphate buffered saline. Any method of combining the adjuvant and the one or more ASFV as described and/or defined herein in the vehicle that improves immunological effectiveness of the immunogenic composition is appropriate.

The volume of a single dose of the compositions and/or immunogenic compositions and/or vaccines as described and/or defined herein may vary but will be generally within the ranges commonly employed in conventional vaccines.

The formulations of the disclosure comprise an effective immunizing amount of the compositions and/or immunogenic compositions and/or vaccines as described and/or defined herein and a physiologically acceptable vehicle. Vaccines comprise an effective immunizing amount of the immunogenic compositions as described and/or defined herein and a physiologically acceptable vehicle. The formulation should fit the mode of administration.

The compositions and/or immunogenic compositions and/or vaccines as described and/or defined herein, if desired, may also contain minor amounts of wetting or emulsifying agents, or pH buffering agents. The compositions and/or immunogenic compositions and/or vaccines as described and/or defined herein can be a liquid solution, suspension, emulsion, tablet, pill, capsule, sustained release formulation, or powder. Oral formulation can include standard carriers such as pharmaceutical grades of mannitol, lactose, starch, magnesium stearate, sodium saccharine, cellulose, magnesium carbonate, etc.

In the context of the present disclosure, the term “a pharmaceutically acceptable or veterinary-acceptable carrier” includes any and all solvents, dispersion media, coatings, adjuvants, stabilizing agents, diluents, preservatives, antibacterial and antifungal agents, isotonic agents, adsorption delaying agents, and the like. In some embodiments, the present disclosure may include lyophilized immunogenic compositions, and stabilizing agents for use in the present disclosure include stabilizers for lyophilization or freeze-drying.

The compositions and/or immunogenic compositions and/or vaccines as described and/or defined herein may be administered by any convenient means. In one embodiment, the administration procedure for recombinant baculovirus or expression products thereof, compositions of the disclosure such as immunological, antigenic or vaccine compositions or therapeutic compositions, can be administered via a parenteral route (e.g., intradermal, intramuscular or subcutaneous). Such an administration enables a systemic immune response, or humoral or cell-mediated responses.

The compositions and/or immunogenic compositions and/or vaccines can be administered alone, or can be co-administered or sequentially administered with compositions, e.g., with other immunological, antigenic or vaccine or therapeutic compositions, thereby providing multivalent or “cocktail” or combination compositions of the disclosure and methods employing them. Again, the ingredients and manner (sequential or co-administration) of administration, as well as dosages can be determined by taking into consideration of such factors as the age, sex, weight, species and condition of the particular host animal, and the route of administration.

### EXAMPLE

Exemplary embodiments of the present disclosure are further described in the following examples, which do not limit the scope of the present disclosure.

### Example 1. Construction and Preparation of Recombinant Viruses

### Plasmid Construction

The nucleotide sequences of the ASFV immunogenic proteins P72 (SEQ ID NO. 9), P54 (SEQ ID NO. 10), CD2v (SEQ ID NO. 11), P30 (SEQ ID NO. 12) and GMCSF (SEQ ID NO. 13) were synthesized by Tools (Tools, Taiwan) with their codon optimized for insect cells, and then cloned into pTriEx-4 plasmids (Novagen, Merck Biosciences, Darmstadt, Germany) The amino acid sequences of the cloned ASFV immunogenic proteins are shown in Table 1 below.

TABLE 1
Amino acid sequences of the cloned
ASFV immunogenic proteins

| | | SEQ |
|---|---|---|
| Protein | Amino acid sequence | ID NO. |
| P72 | EFMASGGAFCLIANDGKADKIILAQDLLNSRISNI | 1 |
| | KNVNKSYGKPDPEPTLSQIEETHLVHFNAHFKPYV | |
| | PVGFEYNKVRPHTGTPTLGNKLTFGIPQYGDFFHD | |
| | MVGHHILGACHSSWQDAPIQGTSQMGAHGQLQTFP | |
| | RNGYDWDNQTPLEGAVYTLVDPFGRPIVPGTKNAY | |
| | RNLVYYCEYPGERLYENVRFDVNGNSLDEYSSDVT | |
| | TLVRKFCIPGDKMTGYKHLVGQEVSVEGTSGPLLC | |
| | NIHDLHKPHQSKPILTDENDTQRTCSHTNPKFLSQ | |
| | HFPENSHNIQTAGKQDITPITDATYLDIRRNVHYS | |
| | CNGPQTPKYYQPPLALWIKLRFWFNENVNLAIPSV | |
| | SIPFGERFITIKLASQKDLVNEFPGLFVRQSRFIA | |
| | GRPSRRNIRFKPWFIPGVINEISLTNNELYINNLF | |
| | VTPEIHNLFVKRVRFSLIRVHKTQVTHTNNNHHDE | |
| | KLMSALKWPIEYMFIGLKPTWNISDQNPHQHRDWH | |
| | KFGHVVNAIMQPTHHAEISFQDRDTALPDACSSIS | |
| | DISPVTYPITLPIIKNISVTAHGINLIDKFPSKFC | |
| | SSYIPFHYGGNAIKTPDDPGAMMITFALKPREEYQ | |
| | PSGHINVSRAREFYISWDTDYVGSITTADLVVSAS | |
| | AINFLLLQNGSAVLRYSTGS | |
| P54 | DSEFFQPVYPRHYGECLSPVTTPSFFSTHMYTILI | 2 |
| | AIVVLVIIIIVLIYLFSSRKKKAAAIEEEDIQFIN | |
| | PYQDQQWVEVTPQPGTSKPAGATTASVKGKPVTGR | |
| | PATNRPATNKPVTDNPVTDRLVMATGGPAAAPAAA | |
| | SAPAHPAEPYTTVTTQNTASQTMSAIENLRQRNTY | |
| | THKDLENSL | |
| CD2v | IDYWVSFNKTIILDSNITNDNNDINGVSWNFFNNS | 3 |
| | FNTLATCGKAGNFCECSNYSTSIYNITNNCSLTIF | |
| | PHNDVFDTTYQVVWNQIINYTIKLLTPATPPNITY | |
| | NCTNFLITCKKNNGTNTNIYLNINDTFVKYTNESI | |
| | LEYNWNNSNINNFTATCIINNTISTSNETTLINCT | |
| | YLTLSSNYFYTFFKLYYIPLSIIIGITISILLISI | |
| | ITFLSLRKRKKHVEEIESPPPESNEEEQCQHDDTT | |
| | SIHEPSPREPLLPKPYSRYQYNTPIYYMRPSTQPL | |
| | NPFPLPKPCPPPKPCPPPKPCPPPKPCPSAESYSP | |
| | PKPLPSIPLLPNIPPLSTQNISLIHVDRII | |
| P30 | EFMDFILNISMKMEVIFKTDLRSSSQVVFHAGSLY | 4 |
| | NWFSVEIINSGRIVTTAIKTLLSTVKYDIVKSARI | |
| | YAGQGYTEHQAQEEWNMILHVLFEEETESSASSEN | |
| | IHEKNDNETNECTSSFETLFEQEPSSEVPKDSKLY | |
| | MLAQKTVQHIEQYGKAPDFNKVIRAHNFIQTIYGT | |
| | PLKEEEKEVVRLMVIKLLKKKGS | |
| GMCSF | APTRPPSPVTRPWQHVDAIKEALSLLNNSNDTAAV | 5 |
| | MNETVDVVCEMFDPQEPTCVQTRLNLYKQGLRGSL | |
| | TRLKSPLTLLAKHYEQHCPLTEETSCETQSITFKS | |
| | FKDSLNKFLFTIPFDCWGPVKK | |

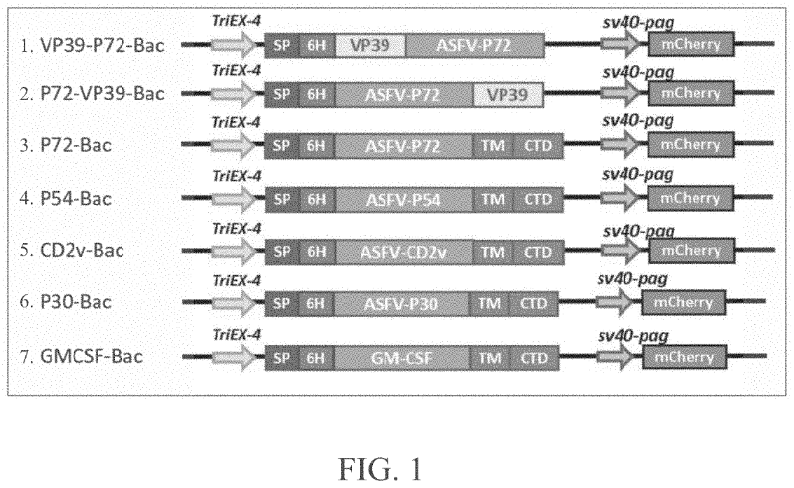

The pTriEx-4 plasmid contains tripartite p10, cytomegalovirus (CMV) and T7 promoters for the convenient expression in insect, mammalian, and bacterial cells. The P72, P54, CD2v, P30 and GMCSF genes were driven by the TriEx promoter, followed by the HM signal protein (honeybee melittin signal peptide) and 6×His-tag in the pTriEx-4 plasmids, to produce plasmids pTriEx-VP39-P72, pTriEx-P72-VP39, pTriEx-P72, pTriEx-P54, pTriEx-CD2v, pTriEx-P30, and pTriEx-GMCSF, respectively (FIG. 1). Among these, the pTriEx-VP39-P72 and pTriEx-P72-VP39 containing the codon optimized full-length P72 and AcMNPV (Autographa californica multiple nucleopolyhedrovirus) derived-VP39 (SEQ ID NO. 14) genes were for the generation of capsid-fused recombinant baculovirus, VP39-P72-Bac and P72-VP39-Bac. The pTriEx-P72, pTriEx-P54, pTriEx-CD2v, pTriEx-P30 and pTriEx-GMCSF contain the codon optimized P72, P54, CD2v, P30 and GMCSF genes associated with the GP64 transmembrane domain (TM) (SEQ ID NO. 15) and the GP64 cytoplasmic domain (CTD) (SEQ ID NO. 16) for membrane anchoring. The amino acid sequences of the cloned VP39, GP64 transmembrane domain (TM) and GP64 cytoplasmic domain (CTD) are shown in Table 2 below.

TABLE 2
Amino acid sequences of the cloned VP39,
GP64 transmembrane domain
(TM) and GP64 cytoplasmic domain (CTD)

| | | SEQ |
|---|---|---|
| | | ID |
| Protein | Amino acid sequence | NO. |
| VP39 | ALVPVGMAPRQMRVNRCIFASIVSFDACI | 6 |
| | TYKSPCSPDAYHDDGWFICNNHLIKRFKM | |
| | SKMVLPIFDEDDNQFKMTIARHLVGNKER | |
| | GIKRILIPSATNYQDVFNLNSMMQAEQLI | |
| | FHLIYNNENAVNTICDNLKYTEGFTSNTQ | |
| | RVIHSVYATTKSILDTTNPNTFCSRVSRD | |
| | ELRFFDVTNARALRGGAGDQLFNNYSGFL | |
| | QNLIRRAVAPEYLQIDTEELRFRNCATCI | |
| | IDETGLVASVPDGPELYNPIRSSDIMRSQ | |
| | PNRLQIRNVLKFEGDTRELDRTLSGYEEY | |
| | PTYVPLFLGYQIINSENNFRLNDFIPRAN | |
| | PNATLGGGAVAGPAPGVAGEAGGGIAV | |
| GP64 | FMFGHVVNFVIILIVILFLY | 7 |
| transmembrane | | |
| domain (TM) | | |
| GP64 | CMIRNRNRQY | 8 |
| cytoplasmic | | |
| domain (CTD) | | |

All the plasmid constructs were inserted with a mCherry fluorescent protein gene driven by the sv40-pag promoter as a reporter. The mCherry gene was driven by the binary sv40-pag promoter for emitting reporter fluorescence in Sf21 and mammalian cells. The plasmids were constructed according to the instructions' manual of In-Fusion® HD Cloning Kit (Clontech Laboratories Inc, CA, USA). Plasmids pTriEx-VP39-P72, pTriEx-P72-VP39, pTriEx-P72, pTriEx-P54, pTriEx-CD2v, pTriEx-P30 and pTriEx-GMCSF were thereby obtained, respectively.

### Recombinant Baculovirus Preparation

pTriEx-VP39-P72, pTriEx-P72-VP39, pTriEx-P72, pTriEx-P54, pTriEx-CD2v, pTriEx-P30 and pTriEx-GMCSF plasmids were co-transfected with FlashBAC™ (Mirus, WI, USA) DNA into Sf21 cells by Cellfectin (Life Technologies, CA, USA) to generate recombinant baculoviruses, VP39-P72-Bac, P72-VP39-Bac, P72-Bac, P54-Bac, CD2v-Bac, P30-Bac and GMCSF-Bac. The expression of mCherry gene product and 6×His-tag are used to trace proper viral infection and protein expression.

### Virus Titer Determination (by 50% Tissue Culture Infection Dose, TCID50)

Sf21 cells (4×10⁴) were seeded in a 96-well plate and incubated at room temperature (26° C.) at least 1 to 2 hours for complete attachment. The virus solutions from recombinant baculoviruses preparation were end-point diluted into different concentrations (10⁻¹˜10⁻¹⁰) with 10% fetal bovine serum (FBS) containing TC-100 insect medium. The medium in each well of the Sf21 cells seeded plate was replaced with 100 μL of the virus solution from each dilution. Each dilution was repeated for eight times. For efficient virus infection, plates were centrifuged at 2000 rpm for 30 min, and placed in 26° C. incubator for four to five days before observation. The virus titer was then determined by calculating the number of infected wells under each dilution of virus.

The VP39-P72-Bac, P72-VP39-Bac, P72-Bac, P54-Bac, CD2v-Bac, P30-Bac and GMCSF-Bac virus clones with high titers were selected and used for recombinant baculoviruses production.

### Example 2. Expression of ASFV Proteins and Fusion Proteins by Recombinant Viruses

To confirm the expression of ASFV proteins by the recombinant viruses in the cells, western blotting analysis was carried out. Specifically, after propagating recombinant baculoviruses VP39-P72-Bac, P72-VP39-Bac, P72-Bac, P54-Bac, CD2v-Bac, P30-Bac and GMCSF-Bac in the Sf21 cells, the Sf21 cells were lysed and analyzed by western blotting for evaluating the expressions of VP39-P72, P72-VP39, P72, P54, CD2v, P30 and GMCSF proteins.

For western blotting analysis, cell lysates were collected and boiled in Laemmli Sample Buffer (TOOLS TAAR-TB2, Taiwan) for 10 minutes, and then loaded into gradient sodium dodecyl sulfate (SDS)-polyacrylamide electrophoresis gel (HR gradient gel solution, TOOLS, Taiwan). Samples were resolved in 10% SDS-PAGE in the running buffer (200 mM glycine, 1% SDS, 2.5 mM Tris/HCl). After resolving, samples were transferred to a polyvinylidene difluoride (PVDF) membrane by using a transfer buffer (25 mM Tris, 192 mM glycine, 10% methanol) at 300 mA for 90 minutes at 4° C. The PVDF membrane containing protein samples was washed briefly in phosphate buffered saline (PBS) and blocked by 5% skimmed milk in PBS for an hour at room temperature. The protein samples were detected by the specific antibody. The protein signals were detected by using mouse anti-6×His-tag monoclonal antibody (1:5000 dilution, EnoGene, NY, USA). Then, the goat anti-mouse IgG conjugated to horseradish peroxidase (HRP) (1:5000 dilution, Invitrogen, CA, USA) was used as the secondary antibody for signal detection. The protein bands were detected by using the Clarity™ Western ECL Blotting Substrates (Bio-Rad) using Classic Blue Autoradiography film BX (Life Science, MO, USA).

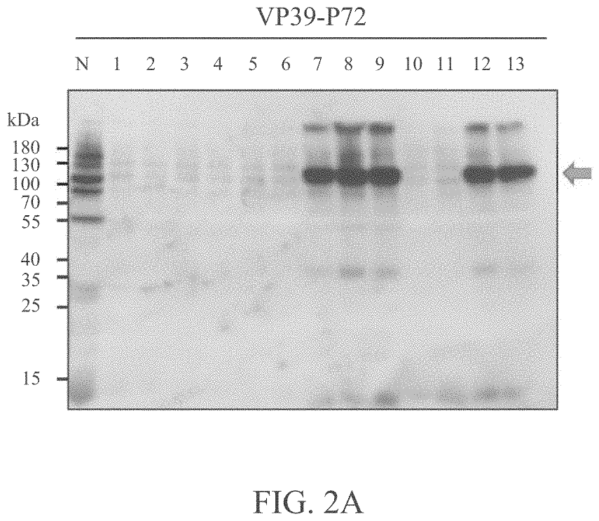

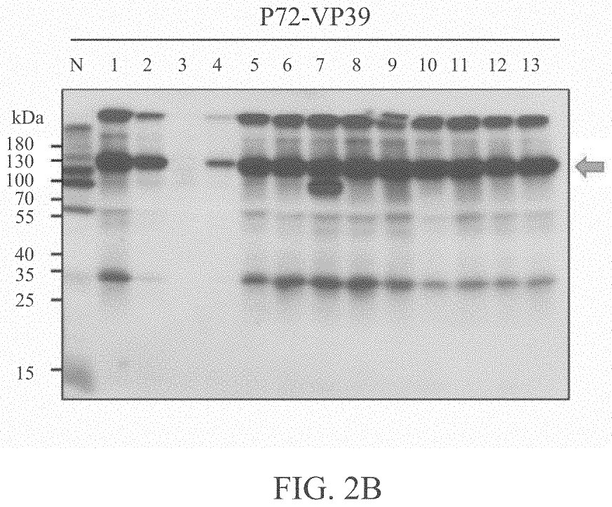

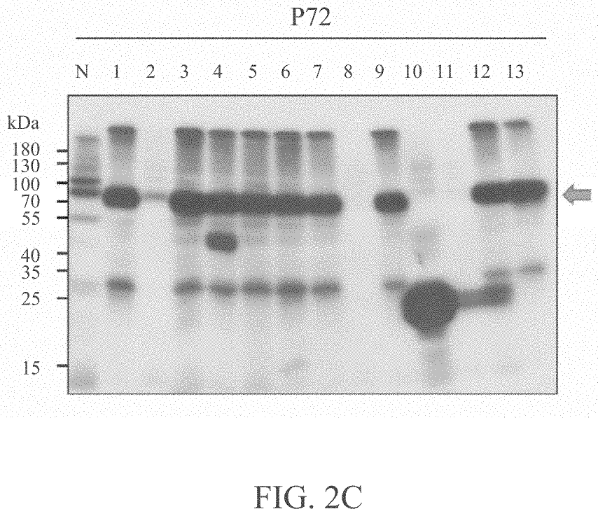

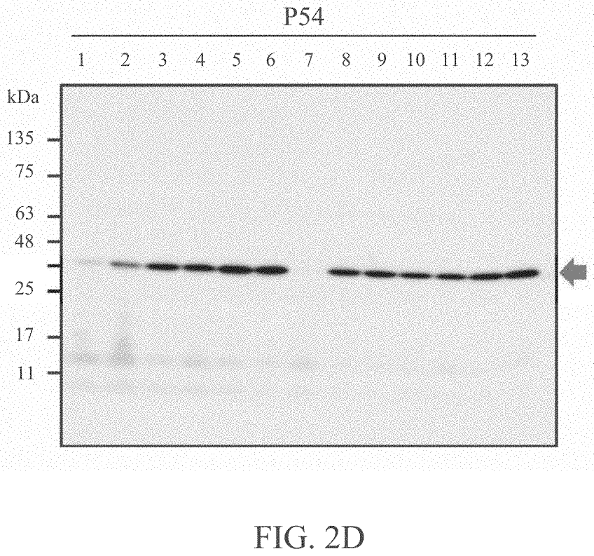

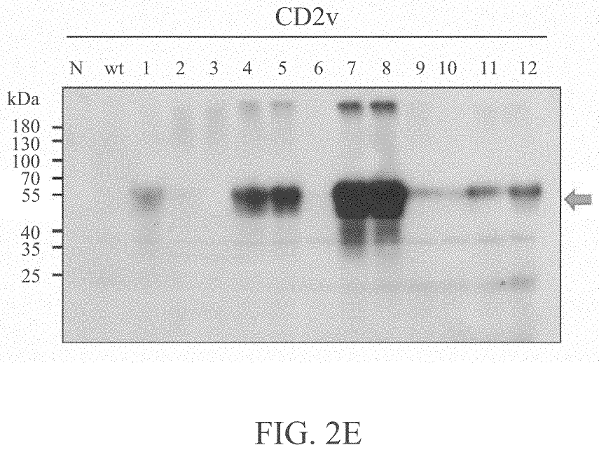

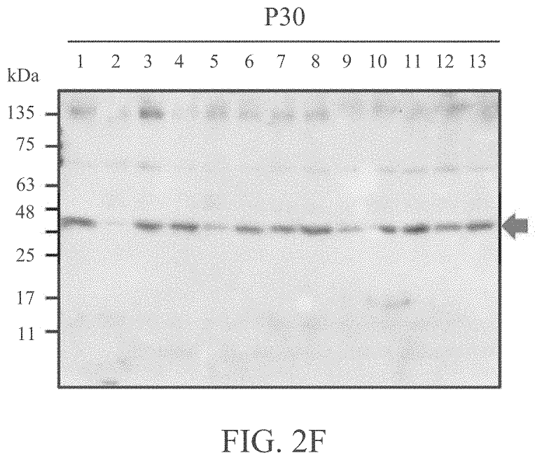

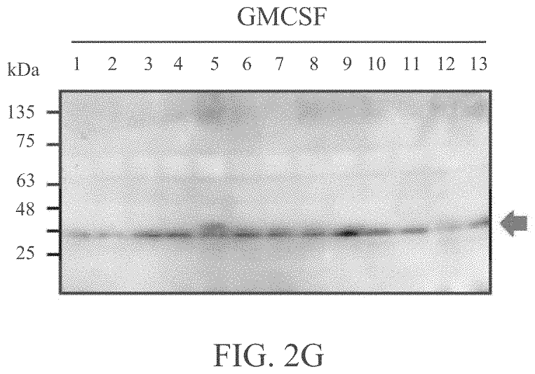

The positive signals of the VP39-P72, P72-VP39, P72, P54, CD2v, P30 and GMCSF proteins expressed by the individual single virus clones derived from VP39-P72-Bac (FIG. 2A), P72-VP39-Bac (FIG. 2B), P72-Bac (FIG. 2C), P54-Bac (FIG. 2D), CD2v-Bac (FIG. 2E), P30-Bac (FIG. 2F), and GMCSF-Bac (FIG. 2G) were observed at the sizes around 115 kDa, 115 kDa, 74 kDa, 30 kDa, 50 kDa, 35 kDa and 30 kDa, respectively. As a negative control, no detectable signal was observed in the lysate of the Sf21 cells infected with wild-type AcMNPV virus.

### Example 3. ASFV Proteins were Displayed on the Surface of the Cells Infected with Recombinant Baculoviruses

The cells infected with recombinant baculoviruses display the ASFV proteins on their cell surfaces and can be used to deliver the cell-based ELISA (enzyme-linked immunosorbent assay) for detecting ASFV viruses.

To confirm the displaying of the ASFV proteins on the surface of the cells infected with recombinant baculoviruses, immunofluorescence assay was carried out. Specifically, Sf21 cells (2×10⁵) were seeded into 8-well Millicell® EZ slides (Millipore), and the cells were transduced with recombinant baculoviruses using a multiplicity of infection (MOI) of 1. The slides were centrifuged at 2,000 rpm for 30 min at room temperature and then incubated at 26° C. for 48 hpi (hours post infection) as indicated. The cells were then fixed with 4% paraformaldehyde, and then blocked with 3% bovine serum albumin (BSA) for 1 h. The cells were then incubated with primary antibody overnight at 4° C. The protein signals were detected by using mouse anti-6×His-tag monoclonal antibody (1:5000 dilution, EnoGene, NY, USA). After overnight incubation, the cells were washed three times with DPBST (Dulbecco's phosphate-buffered saline, DPBS, plus 0.1% Tween 20) and incubated with 1:200 dilutions of Alexa Fluor 488 goat anti-mouse IgG secondary antibody (Invitrogen). Images were obtained with a Zeiss laser confocal microscope (LSM780) using a Fluor 63×/1.40 NA oil-immersion objective. All images were acquired using 1024×1024 diameter pixels, and fluorescence intensity was analyzed by ZEN 2010 software (Zeiss).

The result showed that recombinant baculoviruses P72-VP39-Bac, P54-Bac, P30-Bac and CD2v-Bac used to infect the Sf21 cells at 3 days post infection with an MOI of 1 expressed the virus proteins and separately displayed the P72-VP39, P54, P30 and CD2v proteins on the cell surface.

### Example 4. Electron Microscopic Examination of the Virus Proteins Displayed on the Surface of Recombinant Baculoviruses P72-VP39-Bac, P54-Bac, P30-Bac, and CD2v-Bac

Supernatants were collected from the P72-VP39-Bac-inoculated, P54-Bac-inoculated, P30-Bac-inoculated and CD2v-Bac-inoculated Sf21 cells. The cell debris was coarsely removed by centrifugation at 10,000 rpm for 30 min, and then the supernatants were collected and subjected onto the 25% (w/w) sucrose cushion in SW28 tubes (Beckman, CA, USA) for centrifugation at 24,000 rpm for 80 min at 4° C. to obtain the viral pellet. After discarding the supernatant, the viral pellets were resuspended with 1 mL PBS, and then subjected to a 25% to 60% (w/w) sucrose gradient at 28,000 rpm for 3 hours. Viral particles were collected and washed with PBS to remove sucrose. These purified viral particles were then fixed, labeled with anti-His immunogold, and visualized by electron microscopy (EM) with negative staining as described in the art. Briefly, an aliquot of 10 μL virus particle preparation was loaded onto a carbon-coated grid, letting standstill for 5 min. Grids were then stained with 2% of phosphotungstic acid (PTA) for 1 min, and then, the excess PTA was drained and completely dry-out. The grids were examined directly under EM.

For immunogold labeling, virus particles were loaded onto a collodion-coated EM grid for 5 min. After the removal of excess viral particles by gently blotting with a filter paper at the edge of the grid, an anti-His tag antibody (Invitrogen) was added onto the grid and incubated for 1 hr at room temperature. Grids then underwent 10 second wash for six times in PBS at room temperature, and were incubated with 6 nm gold-conjugated anti-mouse IgG for 1 hr. After six times of washes in PBS, the grids were stained with 2% PTA for 1 min, drained and dry-out, and then examined under the EM.

The result showed that the expression of P54, P30 and CD2v proteins by the plasmids and recombinant baculoviruses of this disclosure were localized and displayed on the baculovirus, and the virus protein P72 is expressed with VP39 at the baculovirus capsid.

### Example 5. Cell-Based ELISA for Detecting ASFV Immunogens

The recombinant baculoviruses P72-Bac, VP39-P72-Bac, P72-VP39-Bac, P54-Bac and P30-Bac were used to separately infect and display P72, P54, and P30 proteins on the surface of the Sf21 cells. These cells were fixed by 4% paraformaldehyde and permeabilized by 0.2% Triton treatment, before determining antibody recognition of a His-tag antibody. Then, the infected-cell-coated plates were washed three times with 100 μL of PBST (PBS containing 0.05% Tween 20) and incubated for 1 h at room temperature with the His-tag antibody. After washing, 100 μL of the goat anti-mouse IgG conjugated to HRP (1:5000 dilution, Invitrogen, CA, USA) was used as the secondary antibody with incubation of 1 hr, followed by reacting with the 3,3′,5,5′-tetramethylbenzidine (TMB) substrate for signal detection.

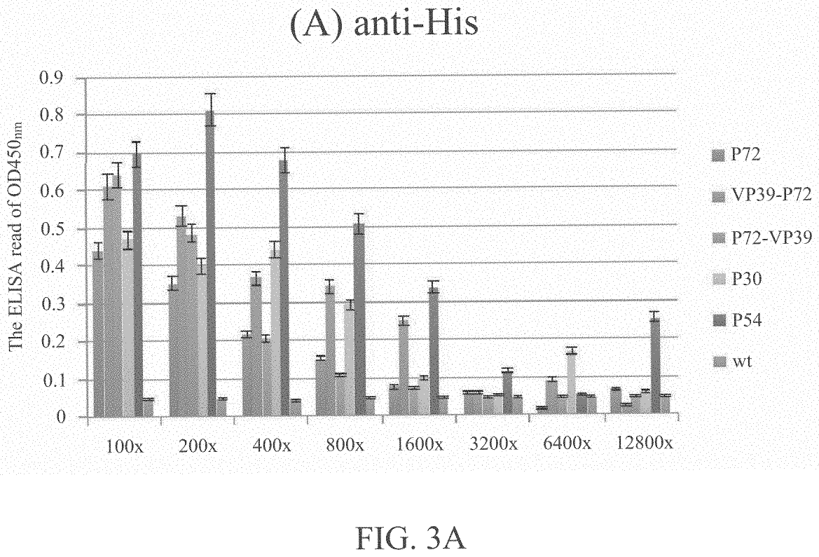

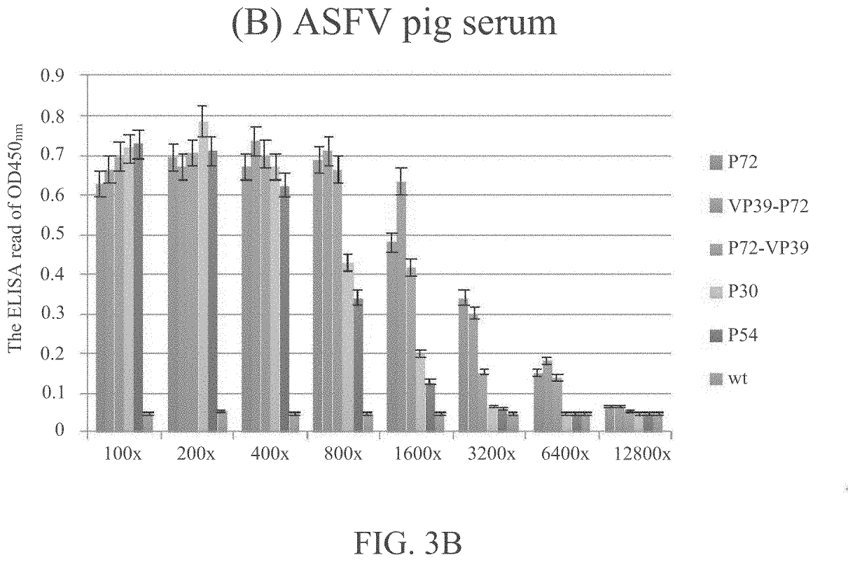

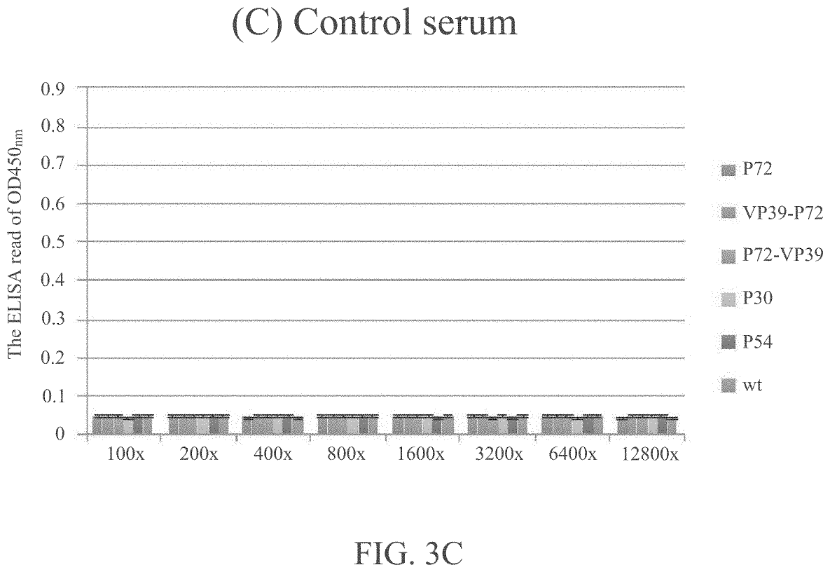

The cells were subjected to hybridization with different dilutions of the anti-His antibody, anti-ASFV antiserum or control serum. The anti-ASFV antiserum was provided by Dr. Linda Dixon from the collaborating lab, the Pirbright Institute, and was obtained from the ASFV infected pig, and therefore contained antibodies to the ASFV. The control serum was provided by Dr. Hui-Wen Chang from the School of Veterinary Medicine, National Taiwan University, and was obtained from the healthy animals. It was found that these virus proteins displaying on the cell surface can be recognized by anti-His antibodies (FIG. 3A) and anti-ASFV antiserum (FIG. 3B), and exhibited yellow colors as positive signals, but not the control serum (FIG. 3C). This indicates that cell-based ELISA utilizing the recombinant baculoviruses of this disclosure to display the virus proteins on cell surface can be used with the antisera from animals infected by the ASFV and detect the presence of the ASFV immunogens.

The present disclosure has been described with embodiments thereof, and it is understood that various modifications, without departing from the spirit of this disclosure, are in accordance with the embodiments of the present disclosure. Hence, the embodiments described are intended to cover the modifications within the scope and the spirit of the present disclosure, rather than to limit the present disclosure. The scope of the claims therefore should be accorded the broadest interpretation so as to encompass all such modifications.

What is claimed is:

1. A recombinant baculovirus having at least one of African swine fever virus (ASFV) proteins P72, P49, PE120R, P54, P30 and CD2v or a fragment thereof, or a combination thereof, wherein the ASFV protein is fused with a baculovirus protein or a fragment thereof.

2. The recombinant baculovirus according to claim 1, wherein the baculovirus protein fused with the ASFV protein is a capsid protein or an envelope protein.

3. The recombinant baculovirus according to claim 2, wherein the baculovirus protein fused with the ASFV protein is VP39, Fusion (F) protein or GP64.

4. The recombinant baculovirus according to claim 1, wherein the ASFV protein or the fragment thereof is displayed on a surface of the recombinant baculovirus.

5. The recombinant baculovirus according to claim 4, wherein the surface is a baculoviral capsid or envelope.

6. The recombinant baculovirus according to claim 5, wherein at least one of the ASFV proteins P72, P49 and PE120R is displayed on the surface of the baculoviral capsid.

7. The recombinant baculovirus according to claim 5, wherein at least one of the ASFV proteins P54, P30 and CD2v is displayed on the surface of the baculoviral envelope.

8. An immunological composition comprising at least one of the recombinant baculovirus of claim 1 and a pharmaceutically acceptable carrier thereof.

9. The immunological composition according to claim 8, further comprising an adjuvant.

10. The immunological composition according to claim 9, wherein the adjuvant is a recombinant baculovirus expressing granulocyte-macrophage colony-stimulating factor (GMCSF), chemokine C-C motif ligand 25 (CCL25) or chemokine C-C motif ligand 29 (CCL29).

11. A method for inducing an immunological response against an African swine fever virus in a host in need thereof, comprising administering to the host the recombinant baculovirus of claim 1.

12. A cell infected with the recombinant baculovirus of claim 1.

13. A method for detecting an African swine fever virus in a host thereof, comprising detecting formation of a complex between the cell of claim 12 and a sample obtained from the host.

14. The method according to claim 13, wherein the detection is performed by immunoassay, counter immuno-electrophoresis, radioimmunoassay, radioimmunoprecipitation assay, enzyme-linked immunosorbent assay, dot blot assay, inhibition of competition assay or sandwich assay.

15. The method according to claim 13, wherein the host is a porcine.

16. The method according to claim 13, wherein the sample is serum.

17. The method according to claim 16, wherein the serum is antiserum.

18. A kit comprising any one of the ASFV proteins of claim 1 and a reagent for immunological detection.

19. A kit comprising the cell of claim 12 and a reagent for immunological detection.

20. A cloning vector comprising a nucleic acid sequence for coding any one of the ASFV proteins of claim 1.


SEQUENCE LISTING (16 sequences)

SEQ ID NO: 1 - protein, 650 aa, African swine fever virus
EFMASGGAFCLIANDGKADKIILAQDLLNSRISNIKNVNKSYGKPDPEPTLSQIEETHLVHFNAHFKPYVPVGFEYNKVRPHTGTPTLGNKLTFGIPQYGDFFHDMVGHHILGACHSSWQDAPIQGTSQMGAHGQLQTFPRNGYDWDNQTPLEGAVYTLVDPFGRPIVPGTKNAYRNLVYYCEYPGERLYENVRFDVNGNSLDEYSSDVTTLVRKFCIPGDKMTGYKHLVGQEVSVEGTSGPLLCNIHDLHKPHQSKPILTDENDTQRTCSHTNPKFLSQHFPENSHNIQTAGKQDITPITDATYLDIRRNVHYSCNGPQTPKYYQPPLALWIKLRFWFNENVNLAIPSVSIPFGERFITIKLASQKDLVNEFPGLFVRQSRFIAGRPSRRNIRFKPWFIPGVINEISLTNNELYINNLFVTPEIHNLFVKRVRFSLIRVHKTQVTHTNNNHHDEKLMSALKWPIEYMFIGLKPTWNISDQNPHQHRDWHKFGHVVNAIMQPTHHAEISFQDRDTALPDACSSISDISPVTYPITLPIIKNISVTAHGINLIDKFPSKFCSSYIPFHYGGNAIKTPDDPGAMMITFALKPREEYQPSGHINVSRAREFYISWDTDYVGSITTADLVVSASAINFLLLQNGSAVLRYSTGS

SEQ ID NO: 2 - protein, 183 aa, African swine fever virus
DSEFFQPVYPRHYGECLSPVTTPSFFSTHMYTILIAIVVLVIIIIVLIYLFSSRKKKAAAIEEEDIQFINPYQDQQWVEVTPQPGTSKPAGATTASVGKPVTGRPATNRPATNKPVTDNPVTDRLVMATGGPAAAPAAASAPAHPAEPYTTVTTQNTASQTMSAIENLRQRNTYTHKDLENSL

SEQ ID NO: 3 - protein, 345 aa, African swine fever virus
IDYWVSFNKTIILDSNITNDNNDINGVSWNFFNNSFNTLATCGKAGNFCECSNYSTSIYNITNNCSLTIFPHNDVFDTTYQVVWNQIINYTIKLLTPATPPNITYNCTNFLITCKKNNGTNTNIYLNINDTFVKYTNESILEYNWNNSNINNFTATCIINNTISTSNETTLINCTYLTLSSNYFYTFFKLYYIPLSIIIGITISILLISIITFLSLRKRKKHVEEIESPPPESNEEEQCQHDDTTSIHEPSPREPLLPKPYSRYQYNTPIYYMRPSTQPLNPFPLPKPCPPPKPCPPPKPCPPPKPCPSAESYSPPKPLPSIPLLPNIPPLSTQNISLIHVDRII

SEQ ID NO: 4 - protein, 198 aa, African swine fever virus
EFMDFILNISMKMEVIFKTDLRSSSQVVFHAGSLYNWFSVEIINSGRIVTTAIKTLLSTVKYDIVKSARIYAGQGYTEHQAQEEWNMILHVLFEEETESSASSENIHEKNDNETNECTSSFETLFEQEPSSEVPKDSKLYMLAQKTVQHIEQYGKAPDFNKVIRAHNFIQTIYGTPLKEEEKEVVRLMVIKLLKKKGS

SEQ ID NO: 5 - protein, 127 aa, Artificial Sequence
  Partial GMCSF sequence
APTRPPSPVTRPWQHVDAIKEALSLLNNSNDTAAVMNETVDVVCEMFDPQEPTCVQTRLNLYKQGLRGSLTRLKSPLTLLAKHYEQHCPLTEETSCETQSITFKSFKDSLNKFLFTIPFDCWGPVKK

SEQ ID NO: 6 - protein, 346 aa, Artificial Sequence
  Baculovirus VP39 partial sequence
ALVPVGMAPRQMRVNRCIFASIVSFDACITYKSPCSPDAYHDDGWFICNNHLIKRFKMSKMVLPIFDEDDNQFKMTIARHLVGNKERGIKRILIPSATNYQDVFNLNSMMQAEQLIFHLIYNNENAVNTICDNLKYTEGFTSNTQRVIHSVYATTKSILDTTNPNTFCSRVSRDELRFFDVTNARALRGGAGDQLFNNYSGFLQNLIRRAVAPEYLQIDTEELRFRNCATCIIDETGLVASVPDGPELYNPIRSSDIMRSQPNRLQIRNVLKFEGDTRELDRTLSGYEEYPTYVPLFLGYQIINSENNFLRNDFIPRANPNATLGGGAVAGPAPGVAGEAGGGIAV

SEQ ID NO: 7 - protein, 20 aa, Artificial Sequence
  GP64 transmembrance domain
FMFGHVVNFVIILIVILFLY

SEQ ID NO: 8 - protein, 10 aa, Artificial Sequence
  GP64 cytoplasmic domain
CMIRNRNRQY

SEQ ID NO: 9 - DNA, 1950 bp, African swine fever virus
gaattcatggctagcggtggtgctttctgtctgatcgctaacgacggcaaggctgataagatcatcctggctcaagatctgctgaactcccgtatctcaaacatcaagaacgttaacaagtcatacggaaagcctgaccctgaaccaaccctgtcacaaatcgaagaaacacacctggtgcacttcaacgctcacttcaagccatacgtccccgtgggtttcgaatacaacaaggttcgccctcacacaggtacccctaccttgggaaacaaactgactttcggaatccctcagtacggtgacttcttccacgacatggtgggtcaccacattctcggagcctgccacagctcatggcaggatgctccaattcaaggtacctcccagatgggtgctcacggccagttgcaaactttcccaagaaacggttacgattgggacaaccaaacacctttggaaggtgccgtctacactctggttgatcccttcggcagaccaatcgttcctggtacaaagaacgcctaccgtaatctcgtttactactgtgaataccctggtgaacgcctgtacgaaaacgttcgttttgacgtgaacggtaactctctggatgaatactcaagcgacgtgacaactctcgttcgcaagttctgtatcccaggcgacaagatgacaggttacaagcacctggttggccaagaagtgtccgttgaaggaacttctggtcctctgttgtgtaacatccacgacctccacaagcctcatcagtctaagcctatcctgactgatgaaaacgacactcagagaacatgtagccacacaaaccctaagtttctgtctcaacacttccctgagaatagccacaacatccaaacagctggaaagcaagacatcacacctatcactgacgccacctacctggacatcaggcgtaacgtgcactactcttgtaacggtccacaaactcctaagtactaccagcctcctctggctctctggatcaagctgcgtttctggttcaacgaaaacgtgaacctggccatcccctctgtttcaatccctttcggtgaacgcttcatcacaatcaagctcgcttcccagaaggatctggttaacgagttccctggactgttcgttcgtcagtcccgtttcatcgctggccgtccatctcgtcgcaacatccgtttcaagccttggttcatccctggagttatcaacgagatctccctgactaacaacgaactgtacatcaacaacctgttcgtcactccagaaatccacaacctgttcgttaagcgcgttcgtttctcactgatcagggttcacaagactcaagtgactcacactaacaacaaccaccacgatgaaaagctgatgtcagccctcaagtggccaatcgaatacatgttcatcggtctgaagcctacatggaacatcagcgaccaaaaccctcaccaacaccgtgactggcacaagttcggtcacgttgtcaacgctatcatgcaacccactcaccacgctgaaatctctttccaggatcgtgacacagctctgccagacgcttgtagctcaatctcagacatctcacctgttacttaccccatcacactccctatcattaagaacattagcgttacagctcacggcatcaacctgatcgataagttcccatctaagttctgctccagctacatccccttccactacggaggtaacgccatcaagactcctgatgaccctggtgctatgatgatcacattcgccctgaagccacgtgaagaataccagcctagcggacacattaacgtgagccgcgctcgtgagttctacatctcatgggacactgattacgttggttccatcacaaccgctgacctggttgtttcagcttcagctatcaacttcctgctgctgcagaacggatctgccgtcctgcgttactctacaggatcc

SEQ ID NO: 10 - DNA, 549 bp, African swine fever virus
gactccgaattcttccagcctgtctacccccgccactacggtgagtgcctgagccctgtcactactccttccttcttctccactcacatgtacaccatcctgatcgctatcgtcgtgctggtcattatcatcatcgtcctgatctacctgttcagcagccgcaagaagaaggccgctgccatcgaagaagaagacatccagttcatcaacccttaccaggaccagcagtgggtggaagtcacccctcagcctggtacctccaagcccgccggcgctactactgccagcgtcggtaaacctgtcaccggtcgccctgctaccaaccgtcctgctaccaataagcctgtgaccgacaaccccgtgaccgaccgtctggtcatggccactggcggtcccgctgccgctccagctgctgctagcgctcctgctcaccctgctgaaccttacaccactgtgaccacccagaacaccgcttcccagactatgagcgctatcgagaacctgcgccagcgtaacacttacacccacaaggacctggaaaacagcctg

SEQ ID NO: 11 - DNA, 1077 bp, African swine fever virus
atcatcctgatcttcctgatcttctccaacatcgtgctgtccatcgactactgggtctccttcaacaagactatcatcctggacagcaacatcaccaacgacaacaacgacatcaacggcgtctcctggaacttcttcaacaacagcttcaacactctggctacctgcggcaaggccggtaacttctgcgagtgcagcaactacagcacttccatctacaacatcactaacaactgctccctgaccatcttcccccacaacgacgtcttcgacactacttaccaggtcgtctggaaccagatcatcaactacaccatcaagctgctgacccccgccactccccctaacatcacctacaactgcaccaacttcctgatcacctgcaagaagaacaacggcactaacaccaacatctacctgaacatcaacgacactttcgtcaagtacaccaacgagtccatcctggaatacaactggaacaacagcaacatcaacaacttcactgctacctgcatcatcaacaacactatcagcacttccaacgaaactaccctgatcaactgcacttacctgaccctgtccagcaactacttctacaccttcttcaagctgtactacatccctctgagcatcatcatcggtatcactatctccatcctgctgatctccatcatcactttcctgagcctgcgtaagcgtaagaagcacgtggaggagatcgagtcccctcctcccgagagcaacgaagaggagcagtgccagcacgacgacaccaccagcatccacgaacctagcccccgcgaacccctgctgcctaagccctacagccgttaccagtacaacactcctatctactacatgcgtccttccacccagcccctgaaccccttccctctgcccaagccctgccccccacctaagccttgcccccctcctaagccctgcccaccccctaagccttgtccttccgccgagagctacagcccccccaagcccctgcccagcatccctctgctgcccaacatccctcctctgtccactcagaacatcagcctgatccacgtggaccgtatcatc

SEQ ID NO: 12 - DNA, 594 bp, African swine fever virus
gaattcatggacttcatcctgaacatctccatgaagatggaagtgatcttcaagaccgacctgcgctcctccagccaggtggtgttccacgctggtagcctgtacaactggttctccgttgaaatcatcaactccggtcgtatcgtgacaactgctatcaagaccctgctgagcactgttaagtacgacatcgttaagagcgctcgtatctacgctggtcagggatacaccgaacaccaggcccaggaagaatggaacatgatcctgcacgtgctgttcgaggaagaaaccgagtccagcgctagcagcgaaaacatccacgaaaagaacgacaacgaaaccaacgaatgtacatcatcattcgaaactctgttcgaacaggagccttcatctgaagtgcctaaggactccaagctgtacatgctggctcaaaagacagttcagcacatcgagcagtacggcaaggctcctgacttcaacaaggttatccgtgcccacaacttcatccagaccatctacggtacccctctgaaggaagaagaaaaggaagtggtgcgtctgatggttatcaagctgctgaagaagaagggatcc

SEQ ID NO: 13 - DNA, 381 bp, Artificial Sequence
  Partial GMCSF nucleotide sequence
gctcctactcgtcctccttctcctgttactcgtccttggcagcacgttgacgctattaaggaagctctgagcctgctgaacaactctaacgacacagctgctgttatgaacgaaactgttgacgtggtttgtgaaatgttcgaccctcaggaacctacatgcgtgcaaactcgtctgaacctgtacaagcagggtctgcgtggtagcctgacacgtctgaagtcccctctgactctgctggctaagcactatgaacagcactgccctctgactgaagaaacatcctgtgaaactcaaagcatcactttcaagtccttcaaggactcactgaacaagttcctgttcactatccctttcgactgttggggtcctgtgaagaag

SEQ ID NO: 14 - DNA, 1038 bp, Artificial Sequence
  Baculovirus VP39 partial sequence
gcgctagtgcccgtgggtatggcgccgcgacaaatgagagttaatcgctgcattttcgcgtccatcgtgtcgttcgacgcgtgcataacatacaaatcgccgtgttcgcccgacgcgtatcatgacgatggatggtttatttgcaacaaccacctcataaaacgttttaaaatgtcaaaaatggttttgcccattttcgacgaagacgacaatcaattcaaaatgacgatcgctaggcatttagttggaaataaagaaagaggtatcaagcgaattttaattccaagcgcaaccaattaccaagacgtgtttaatctaaacagtatgatgcaagccgaacagctaatctttcatttgatatataacaacgaaaacgcagttaacactatatgcgacaatctaaaatataccgaaggtttcacaagcaacacgcaacgcgttatacacagcgtttacgcaactacaaaaagcattctggacaccacaaacccgaacacgttttgttcgcgggtgtcgcgagacgaattgcgtttctttgacgtgaccaacgcccgagcgcttcgaggcggtgctggcgatcaattatttaacaattacagtggatttttgcaaaatttgattcgacgcgcagtagcgcccgagtacttgcaaatcgacacggaggaattgaggtttagaaattgcgccacgtgtataattgacgaaacgggtctggtcgcgtctgtgcccgacggccccgagttgtacaacccgataagaagcagtgacattatgagaagtcaacccaatcgtttgcaaattagaaacgttttgaaatttgaaggcgacacacgtgagctggacagaacgcttagcggatacgaagaatacccgacgtacgttccgctgtttttgggataccaaataatcaattcagaaaacaactttttgcgcaacgactttataccaagagcaaatcctaacgctactctgggcggcggcgcagtggcaggtcctgcgcctggtgttgcaggcgaagcaggtggaggaatagccgtc

SEQ ID NO: 15 - DNA, 60 bp, Artificial Sequence
  GP64 transmembrane domain
ttcatgtttggtcatgtagttaactttgtaattatattaattgtgattttatttttgtac

SEQ ID NO: 16 - DNA, 30 bp, Artificial Sequence
  GP64 cytoplasmic domain
tgtatgattagaaaccgtaatagacaatat